(19) United States
(12) Patent Application Publication
(10) Pub. No.: US 2022/0325298 A1
(43) Pub. Date: Oct. 13, 2022

(54) CRISPR EPIGENETIC THERAPEUTICS FOR PAIN MANAGEMENT

(71) Applicant: University of Pittsburgh - Of the Commonwealth System of Higher Education, Pittsburgh, PA (US)

(72) Inventors: Samira Kiani, Pittsburgh, PA (US); Mo Reza Ebrahimkhani, Pittsburgh, PA (US); Tahere Mokhtari, Pittsburgh, PA (US)

(73) Assignee: University of Pittsburgh - Of the Commonwealth System of Higher Education, Pittsburgh, PA (US)

(21) Appl. No.: 17/717,895

(22) Filed: Apr. 11, 2022

Related U.S. Application Data
(60) Provisional application No. 63/173,892, filed on Apr. 12, 2021.

(51) Int. Cl. C12N 15/86; C12N 9/22; C12N 15/10; C12N 15/11

(52) U.S. Cl. CPC C12N 15/86; C12N 9/22; C12N 15/102; C12N 15/111; C12N 2310/20

(57) ABSTRACT

Disclosed are effective, specific, and durable pain management therapies using Clustered Regularly Interspaced Short Palindromic Repeats (CRISPR-based) epigenetic modulation of endogenous pathways involved in pain.

### CROSS REFERENCE TO RELATED APPLICATION

This patent application claims the benefit of U.S. Provisional Patent Application No. 63/173,892, filed Apr. 12, 2021, the disclosure of which is incorporated by reference in its entirety herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 37,488 Byte ASCII (Text) file named “759_403 ST25.txt,” created Apr. 8, 2022.

### BACKGROUND OF THE INVENTION

Over 20% of U.S. adults experience chronic pain (Centers for Disease Control and Prevention. Prevalence of chronic pain and high-impact chronic pain among adults—United States, 2016, Morbidity and Mortality Weekly Report, 67(36): 1001-1006 (2018)). Chronic pain costs $560 billion annually in medical costs, lost productivity, and disability programs. Few treatments are effective because the experience of pain depends on biological, psychological, and social factors. Many current therapies fall short in targeting multiple pathways at the same time, and efficiency suffers from not addressing the complex etiology of pain.

There is a desire for new pain management therapies.

### BRIEF SUMMARY OF THE INVENTION

An aspect of the invention provides a recombinant herpes simplex virus (HSV) vector comprising one or more polynucleotides encoding (a) at least one guide RNA (gRNA) comprising a guide sequence that hybridizes to a target sequence, and (b) a Cas nuclease.

The HSV vector can comprise one or more modulators (e.g., repressors and/or activators). The one or more repressors can be selected from the group consisting of Hp1a, Krab, MeCP2, and combinations thereof. The one or more activators can be selected from the group consisting of VP64, p65, Rta, HSF1, and combinations thereof.

An aspect of the invention provides a pharmaceutical composition comprising (a) a first herpes simplex virus (HSV) vector comprising one or more polynucleotides encoding a Cas nuclease and (b) a second HSV vector comprising one or more polynucleotides encoding (i) at least one guide RNA comprising a guide sequence that hybridizes to a target sequence and (ii) one or more repressors.

An aspect of the invention provides a pharmaceutical composition comprising (a) a first herpes simplex virus (HSV) vector comprising one or more polynucleotides encoding a Cas nuclease and (b) a second HSV vector comprising one or more polynucleotides encoding (i) at least one guide RNA comprising a guide sequence that hybridizes to a target sequence and (ii) one or more activators.

An aspect of the invention provides a multiplexed CRISPR-based circuit comprising (a) one or more guide RNAs (gRNAs) comprising (i) one or more guide sequences complementary to a portion of target sequence and (ii) one or more gRNA scaffolds; (b) a nucleotide sequence encoding one or more repressors or one or more activators; and (c) a nucleotide sequence encoding a Cas nuclease.

In one aspect, the one or more repressors or activators is fused to an RNA binding protein, and the one or more gRNA scaffolds comprise an aptamer target site specific for the RNA binding protein (e.g., the nucleotide sequences of SEQ ID NOs: 36 and 37). In this respect, the multiplexed CRISPR-based circuit comprises (a) one or more guide RNAs (gRNAs) comprising (i) one or more guide sequences complementary to a portion of target sequence and (ii) one or more gRNA scaffolds comprising an aptamer target site specific for an RNA binding protein; (b) a nucleotide sequence encoding the RNA binding protein fused to one or more repressors or one or more activators; and (c) a nucleotide sequence encoding a Cas nuclease.

An aspect of the invention further provides a method for ameliorating pain in a subject comprising administering to the subject a therapeutically effective amount of the HSV vector, multiplexed CRISPR-based circuit, or pharmaceutical composition thereof, thereby ameliorating pain in the subject.

### BRIEF DESCRIPTION OF THE SEVERAL VIEWS OF THE DRAWINGS

FIGS. 1A and 1B are schematics demonstrating transcription repression (FIG. 1A) or activation (FIG. 1B) using the HSV vector, multiplexed CRISPR-based circuit, or pharmaceutical composition thereof as described in Example 2.

FIGS. 2A and 2B are schematics demonstrating aptamer mediated CRISPR epigenetic modulation in vitro. FIG. 2A shows aptamer-mediated recruitment of effector domains to the CRISPR complex. FIG. 2B shows an experimental design in which mouse neuroblastoma (N2A) cells were transfected with gRNA together with dCas9 plasmid and effector cassette as described in Example 1.

FIGS. 3A and 3B are graphs showing fold changes of mRNA of Scna9 (FIG. 3A) and Myd88 (FIG. 3B) relative to the No Guide (no gRNA) group as described in Example 1.

FIGS. 4A-4C are graphs showing fold changes of mRNA of Penk (FIG. 4A), GAD1 (FIG. 4B), and Kcna2 (FIG. 4C) relative to the No Guide (no gRNA) group as described in Example 1.

### DETAILED DESCRIPTION OF THE INVENTION

Transcriptional control over genes involved in acute and chronic pain can generate a universal therapeutic modality to control pain without the risk of leaving a permanent change in DNA code, as well as unintended side effects of universal small molecules. An aspect of the invention provides an effective, specific, and durable pain management therapy using Clustered Regularly Interspaced Short Palindromic Repeats (CRISPR-based) epigenetic modulation of endogenous pathways involved in pain.

The CRISPR-Cas system, which mediates adaptive immunity within bacteria and archaea, has emerged as a powerful tool for genome engineering. Cas (e.g., Cas9) is an RNA-guided endonuclease that can be directed to specific DNA sequences through complementarity between the Cas-associated guide RNA (gRNA) and the target locus.

In one aspect, the invention provides a recombinant herpes simplex virus (HSV) vector comprising one or more polynucleotides encoding (a) at least one guide RNA (gRNA) comprising a guide sequence that hybridizes to a target sequence and (b) a Cas nuclease.

HSV vectors can accommodate large inserts and are well adapted for persistence in neurons as non-integrating, circular episomes. In particular, the use of HSV (e.g., HSV-1) allows the targeting of nerve cells (e.g., by subcutaneous injection and retrograde transport to neurons), which offers specificity in pain management.

The HSV vector for use in an aspect of the invention can be an HSV-1 vector or an HSV-2 vector. In one aspect, the HSV vector is a HSV-1 vector. The vector can be derived from a wild-type HSV strain or from a laboratory strain (e.g., KOS) or mutant strain. In this context, the vector can be said to be “derived from” a strain by virtue of the mutagenesis of the vector being described with reference to the strain.

HSV is a complex, non-integrating DNA virus capable of infecting a very wide range of human and animal cells. The viral genome contains more than 80 genes and is composed of two unique segments, UL and Us, each flanked by inverted repeats that encode critical diploid genes. An important feature of HSV replication is the expression of its genes in waves referred to as cascade regulation (Rajcani, Virus Genes, 28: 293-310 (2004)). Removal of the essential immediate-early (IE) genes ICP27 and ICP4 renders the virus completely defective and incapable of expression of early (E) genes involved in viral genome replication and late (L) genes functioning in progeny virion assembly. These replication-defective viruses can be grown on complementing cells that express (complement) the missing ICP4 and ICP27 gene products and can then be used to infect non-complementing cells where the viral genome takes residence as a stable nuclear episome. However, vectors that preserve the ICP0 and ICP22 IE genes are toxic to cells, but inactivation or deletion of these genes, the ICP0 gene in particular, hampers transgene expression.

The HSV vector for use in aspects of the invention is not capable of the full aetiology and pathology of the naturally occurring virus, and is rendered incapable in some essential aspect, such as reproduction, toxicity, or transmission. In one aspect, the HSV vector is an attenuated and/or avirulent form. As used herein, “attenuated” means that a replication-competent vector form of the virus is capable of replication, but is less pathogenic. As used herein, “avirulent” means that a replication-defective form of the virus is not capable of a virulent infection in an individual, i.e. where the virus cannot be transferred to another individual by the normal route of infection associated with the relevant DNA virus.

In order to ensure that the virus is attenuated or avirulent, components (e.g., a nucleotide sequence encoding a Cas nuclease) of the HSV vector or multiplexed CRISPR-based circuit of aspects of the invention can be inserted into a location of the HSV genome that encodes a significant function or protein.

Attenuated or avirulent forms of the HSV vector include, but are not limited to, replication-deficient forms of the virus, attenuated forms of the virus, and forms of the virus modified, for example, to prevent integration of the viral genome into the host genome.

The Cas nuclease can be any suitable Cas nuclease. In one aspect, the Cas nuclease is Cas9 nuclease. For example, the Cas9 nuclease can be a catalytically dead variant of Streptococcus pyogenes Cas9 (SpCas9) that includes mutations corresponding to D10A and H840A mutations (corresponding to the nucleotide sequence of SEQ ID NO: 38). In another aspect, the Cas9 nuclease is selected from the following Cas9 variants: Cas9-a2 (corresponding to the nucleotide sequence of SEQ ID NO: 39), Cas9-β2 (corresponding to the nucleotide sequence of SEQ ID NO: 40), and Cas9-α2-β2 (corresponding to the nucleotide sequence of SEQ ID NO: 41). As such, the one or more polynucleotides encoding Cas9 nuclease can comprise the nucleotide sequence of SEQ ID NO: 38, SEQ ID NO: 39, SEQ ID NO: 40, SEQ ID NO: 41, or combinations thereof. Cas9 nuclease and its variants are described in Ferdosi et al., Nature Communications, 10: 1842 (2019). Kiani et al., Nat. Methods, 12(11): 1051-4 (2015) describes that altering Cas9-associated guide RNA (gRNA) correlates to Cas9 nuclease activity.

The HSV vector comprises at least two, at least three, at least four, or at least five guide RNAs (e.g., 2, 3, 4, 5, 6, 7, 8, 9, 10, or ranges of any of these values). The guide RNAs can hybridize to the same or different target sequences.

The guide sequence complementary to a portion of the target sequence can be any suitable length. For example, the guide sequence can be about 10 to about 25 nucleotides (e.g., 10, 11, 12, 13, 14, 15, 16, 17, 18, 19, 20, 21, 21, 22, 23, 24, 25, or ranges of any of these values). In one aspect, the guide sequence is about 14 nucleotides or about 20 nucleotides in length.

The target sequence to which the guide sequence hybridizes can be any suitable target sequence. In a preferred aspect, the target sequence is from a gene involved in nociceptive processing (see Garland, Prim. Care, 39(3): 561-571 (2012)). Exemplary target sequences include, but are not limited to, portions of the genes of SCN9A, MyD88, PENK, GAD1, KCNA2, and combinations thereof. SCN9A encodes voltage-gated sodium-channel type IX a subunit, known as Naᵥ1.7 (see Example 2). MyD88 signaling in dorsal root ganglion (DRG) contributes to persistent neuroinflammation (see Example 2). PENK encodes a preproprotein that is proteolytically processed to generate protein products including the pentapeptide opioids Met-enkephalin and Leu-enkephalin, which are stored in synaptic vesicles, then released into the synapse where they bind to mu- and delta-opioid receptors to modulate the perception of pain (see Example 1). KCNA2 is a voltage-gated potassium channel that mediates transmembrane potassium transport in excitable membranes, primarily in the brain and the central nervous system, but also in the cardiovascular system (see Example 1).

In this respect, the guide sequence can be selected from the group consisting of: (a) one or more of SEQ ID NOs: 1-7 (corresponding to SCN9A); (b) one or more of SEQ ID NOs: 8-12 (corresponding to MyD88); (c) one or more of SEQ ID NOs: 13-20 (corresponding to PENK); (d) one or more of SEQ ID NOs: 21-28 (corresponding to GAD1); (e) one or more of SEQ ID NOs: 29-35 (corresponding to KCNA2); and (f) combinations thereof. The “one or more” sequences refers to 1, 2, 3, 4, 5, 6, 7, 8, 9, 10, or ranges of any of these values.

The guide RNA can additionally comprise one or more (e.g., 1, 2, 3, 4, 5, or ranges thereof) gRNA scaffolds. In one aspect, the one or more gRNA scaffolds comprises an aptamer target site specific for an RNA binding protein. Exemplary gRNA scaffolds include, but are not limited to, the nucleotide sequences of SEQ ID NO: 36 (repression gRNA scaffold comprising two Ms2 stem loops) and SEQ ID NO: 37 (activation gRNA scaffold comprising two PP7 stem loops).

The HSV vector comprising one or more polynucleotides encoding (a) at least one guide RNA comprising a guide sequence that hybridizes to a target sequence, and (b) a Cas nuclease can additionally comprise one or more polynucleotides encoding one or more (e.g., 1, 2, 3, 4, 5, or ranges of these values) modulators (one or more repressors and/or one or more activators).

Suitable CRISPR modulators for use in aspects of the invention are described in, for example, Yeo et al., Nat. Methods, 15(8): 611-616 (2018), and Moghadam et al., Nature Cell Biology, 22: 1143-1154 (2020).

The repressors for use in aspects of the invention include, but are not limited to, Kruppel-associated box (Krab), MeCP2, SIN3A, HDT1, MBD2B, NIPP1, HP1A, and combinations thereof.

In one aspect, the HSV vector comprises one or more polynucleotides encoding a repression gRNA (repression gRNA scaffold) and a repression effector (i.e., repressor). For example, the HSV vector can further comprise the nucleotide sequence of SEQ ID NO: 42 (corresponding to MS2-Hp1a-Krab) or SEQ ID NO: 43 (corresponding to MS2-Krab-MeCP2). The HSV vector also can comprise the nucleotide sequence of SEQ ID NO: 36 (repression gRNA scaffold comprising two Ms2 stem loops).

The activators for use in aspects of the invention include, but are not limited to, VP64, p65, Rta, HSF1, and combinations thereof

In another aspect, the HSV vector comprises one or more polynucleotides encoding an activation gRNA (activation gRNA scaffold) and an activation effector (i.e., activator). For example, the HSV vector can further comprise the nucleotide sequence of SEQ ID NO: 44 (corresponding to PCP-VP64-p65 AD-BRLF1 AD). The HSV vector also can comprise the nucleotide sequence of SEQ ID NO: 37 (activation gRNA scaffold comprising two PP7 stem loops).

The Cas nuclease (e.g., Cas9, dCas9) can be directly fused to the activation or repression domains. Alternatively, the Cas9 nuclease can be separate from the activation or repression domains. For example, the activation and repression domains can be brought to the Cas nuclease (and gRNA) with the use of an RNA binding protein (e.g., MS2 coat protein, such as MS2-Hp1a-Krab corresponding to SEQ ID NO: 42 or MS2-Krab-MeCP2 corresponding to SEQ ID NO: 43). In such a situation, the one or more gRNAs comprise (i) one or more guide sequences complementary to a portion of target sequence and (ii) one or more gRNA scaffolds comprising an aptamer target site specific for the RNA binding protein (e.g., repression gRNA scaffold comprising two Ms2 stem loops corresponding to SEQ ID NO: 36).

Additionally or alternatively, the one or more polynucleotides encoding (a) at least one guide RNA comprising a guide sequence that hybridizes to a target sequence and (b) a Cas nuclease can be administered in two or more (e.g., 2, 3, 4, 5, or ranges of these values) vectors. In that respect, an aspect of the invention provides a pharmaceutical composition comprising (a) a first HSV vector comprising one or more polynucleotides encoding a Cas nuclease and (b) a second HSV vector comprising one or more polynucleotides encoding (i) at least one guide RNA comprising a guide sequence that hybridizes to a target sequence and (ii) one or more (e.g., 1, 2, 3, 4, 5, or ranges of these values) modulators (e.g., one or more repressors and/or one or more activators). Exemplary modulators are described herein.

In one aspect, the first HSV vector comprises one or more polynucleotides encoding a Cas nuclease and the second HSV vector comprises one or more polynucleotides encoding a repression gRNA (repression gRNA scaffold) and a repression effector (i.e., repressor). For example, the second HSV vector can comprise the nucleotide sequence of SEQ ID NO: 42 (corresponding to MS2-Hp1a-Krab) or SEQ ID NO: 43 (corresponding to MS2-Krab-MeCP2). The second HSV vector also can comprise the nucleotide sequence of SEQ ID NO: 36 (repression gRNA scaffold comprising of two Ms2 stem loops).

In another aspect, the first HSV vector comprises one or more polynucleotides encoding a Cas nuclease and the second HSV vector comprises one or more polynucleotides encoding activation gRNA (activation gRNA scaffold) and an activation effector (i.e., activator). For example, the second HSV vector can comprise the nucleotide sequence of SEQ ID NO: 44 (corresponding to PCP-VP64-p65 AD-BRLF1 AD). The second HSV vector also can comprise the nucleotide sequence of SEQ ID NO: 37 (activation gRNA scaffold comprising two PP7 stem loops).

An aspect of the invention also provides a multiplexed CRISPR-based circuit comprising (a) one or more guide RNAs (gRNAs) comprising (i) one or more guide sequences complementary to a portion of target sequence and (ii) one or more gRNA scaffolds; (b) a nucleotide sequence encoding one or more repressors or one or more activators; and (c) a nucleotide sequence encoding a Cas nuclease. The components of the multiplexed CRISPR-based circuit are described herein.

In one aspect, the one or more repressors or activators is fused to an RNA binding protein, and the one or more gRNA scaffolds comprise an aptamer target site specific for the RNA binding protein (e.g., the nucleotide sequences of SEQ ID NOs: 36 and 37). In this respect, the multiplexed CRISPR-based circuit comprises (a) one or more guide RNAs (gRNAs) comprising (i) one or more guide sequences complementary to a portion of target sequence and (ii) one or more gRNA scaffolds comprising an aptamer target site specific for an RNA binding protein; (b) a nucleotide sequence encoding the RNA binding protein fused to one or more repressors or one or more activators; and (c) a nucleotide sequence encoding a Cas nuclease.

The multiplexed CRISPR-based circuit can be used for simultaneous gene activation and/or repression in vivo to target pain modulation. In this respect, the multiplexed CRISPR-based circuit can contain nucleotide sequences encoding repressor(s) and activator(s).

The components of the multiplexed CRISPR-based circuit can be present in a single vector. Alternatively, one or more vectors can be employed each containing one or more components of the multiplexed CRISPR-based circuit. For example, the nucleotide sequence encoding the Cas nuclease can be packaged in a vector for DNA-based viral delivery (e.g., HSV, such as HSV-1).

Examples of suitable vectors include plasmids (e.g., DNA plasmids), bacterial vectors (e.g., a Listeria or Salmonella vector), yeast vectors, and viral vectors. In one aspect, the vector is a viral vector, such as retrovirus, poxvirus, adenovirus, adeno-associated virus, HSV, polio virus, alphavirus, baculorvirus, and Sindbis virus. In a specific aspect, the vector is a HSV (e.g., HSV-1) vector.

The vectors (e.g., HSV vectors) for use in aspects of the invention can include an expression control sequence operatively linked to coding sequence, such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

The term “enhancer” as used herein, refers to a DNA sequence that increases transcription of, for example, a nucleotide sequence to which it is operably linked. Enhancers can be located many kilobases away from the coding region of the nucleotide sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. For example, the nucleotide encoding the polypeptide can be operably linked to a CMV enhancer/chicken β-actin promoter (also referred to as a “CAG promoter”).

Additionally, the vector can comprise a reporter to identify the transfection/transduction efficiency of the vector.

The vector or multiplexed CRISPR-based circuit can be administered alone or in a composition (e.g., pharmaceutical composition) that can comprise at least one carrier (e.g., a pharmaceutically acceptable carrier), as well as other therapeutic agents (e.g., pain inhibitors).

The pharmaceutically acceptable carrier (or excipient) is preferably one that is chemically inert to the vector or multiplexed CRISPR-based circuit and one that has no detrimental side effects or toxicity under the conditions of use. Such pharmaceutically acceptable carriers include, but are not limited to, water, saline, Cremophor EL (Sigma Chemical Co., St. Louis, Mo.), propylene glycol, polyethylene glycol, alcohol, and combinations thereof. The choice of carrier will be determined in part by the particular vector or multiplexed CRISPR-based circuit, as well as by the particular method used to administer the composition. Accordingly, there is a wide variety of suitable formulations of the composition.

Methods for preparing administrable (e.g., parenterally administrable) vectors, multiplexed CRISPR-based circuits, or pharmaceutical compositions thereof are known or apparent to those skilled in the art.

An aspect of the invention also provides a method of managing pain or ameliorating pain in a subject. The method comprises administering to the subject a therapeutically effective amount of the vector, multiplexed CRISPR-based circuit, or pharmaceutical composition thereof. The therapeutically effective amount refers to an amount of the vector(s), multiplexed CRISPR-based circuit, or pharmaceutical composition thereof that ameliorates, reduces, inhibits, or eliminates pain in the subject. Additionally or alternatively, the therapeutically effective amount refers to an amount of the vector, multiplexed CRISPR-based circuit, or pharmaceutical composition thereof that reduces, inhibits, or eliminates symptoms associated with pain including, but not limited to, inflammation (e.g., neuroinflammation), edema, and hyperalgesia.

The vector, multiplexed CRISPR-based circuit, or pharmaceutical composition thereof can be administered to a subject (e.g., a mammal, such as a non-human mammal including a mouse, rat, guinea pig, hamster, rabbit, cat, dog, pig, cow, horse, or non-human primate, or a human subject) by any suitable route including, but not limited to, parental (subcutaneous, intramuscular, intradermal, intraperitoneal, intrathecal, intravenous, and intratumoral), topical, oral, or local administration.

In one aspect, the vector, multiplexed CRISPR-based circuit, or pharmaceutical composition thereof is administered by subcutaneous injection. The vector(s), multiplexed CRISPR-based circuit, or pharmaceutical compositions thereof preferably targets nerve cells for the management of pain (e.g., the amelioration, reduction, inhibition, or elimination of pain).

When multiple administrations are given, the administrations can be at one or more sites in a host and a single dose can be administered by dividing the single dose into equal portions for administration at one, two, three, four or more sites on the individual.

When the vector, multiplexed CRISPR-based circuit, or pharmaceutical composition thereof is administered with one or more additional therapeutic agents, the vector, multiplexed CRISPR-based circuit, or pharmaceutical compositions thereof and one or more additional therapeutic agents can be coadministered to the mammal. In this regard, the vector, multiplexed CRISPR-based circuit, or pharmaceutical compositions thereof can be administered first and the one or more additional therapeutic agents can be administered second, or vice versa. Alternatively, the HSV vector, multiplexed CRISPR-based circuit, or pharmaceutical compositions thereof and the one or more additional therapeutic agents can be administered simultaneously.

Aspects, including embodiments, of the subject matter described herein may be beneficial alone or in combination, with one or more other aspects or embodiments. Without limiting the foregoing description, certain non-limiting aspects of the disclosure numbered 1-54 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A recombinant herpes simplex virus (HSV) vector comprising one or more polynucleotides encoding (a) at least one guide RNA comprising a guide sequence that hybridizes to a target sequence, and (b) a Cas nuclease.

(2) The HSV vector of aspect 1, wherein the HSV vector is HSV-1.

(3) The HSV vector of aspect 1 or 2, wherein the Cas nuclease is Cas9 nuclease.

(4) The HSV vector of aspect 3, wherein the one or more polynucleotides encoding Cas9 protein comprise the nucleotide sequence of SEQ ID NO: 38, SEQ ID NO: 39, SEQ ID NO: 40, or SEQ ID NO: 41.

(5) The HSV vector of any one of aspects 1-4, wherein the target sequence is selected from the group consisting of SCN9A, MyD88, PENK, GAD1, KCNA2, and combinations thereof.

(6) The HSV vector of any one of aspects 1-5, wherein the guide sequence is selected from the group consisting of:

(a) one or more of SEQ ID NOs: 1-7;

(b) one or more of SEQ ID NOs: 8-12;

(c) one or more of SEQ ID NOs: 13-20;

(d) one or more of SEQ ID NOs: 21-28;

(e) one or more of SEQ ID NOs: 29-35; and

(f) combinations thereof.

(7) The HSV vector of any one of aspects 1-6, wherein HSV vector comprises at least two guide RNAs.

(8) The HSV vector of any one of aspects 1-7, wherein HSV vector comprises at least three guide RNAs.

(9) The HSV vector of any one of aspects 1-8, wherein HSV vector comprises at least four guide RNAs.

(10) The HSV vector of any one of aspects 1-9, wherein HSV vector comprises at least five guide RNAs.

(11) The HSV vector of any one of aspects 7-10, wherein the guide RNAs hybridize to different target sequences.

(12) The HSV vector of any one of aspects 1-11, further comprising one or more polynucleotides encoding one or more repressors.

(13) The HSV vector of aspect 12, wherein the one or more repressors are selected from the group consisting of Hp1a, Krab, MeCP2, and combinations thereof.

(14) The HSV vector of aspect 12 or 13, wherein the one or more polynucleotides encoding one or more repressors comprise the nucleotide sequence of SEQ ID NO: 42 or SEQ ID NO: 43.

(15) The HSV vector of any one of aspects 12-14, further comprising one or more repression gRNA scaffolds.

(16) The HSV vector of aspect 15, wherein the one or more repression gRNA scaffolds comprise the nucleotide sequence of SEQ ID NO: 36.

(17) The HSV vector of any one of aspects 1-11, further comprising one or more polynucleotides encoding one or more activators.

(18) The HSV vector of aspect 17, wherein the one or more activators are selected from the group consisting of VP64, p65, Rta, HSF1, and combinations thereof.

(19) The HSV vector of aspect 17 or 18, wherein the one or more polynucleotides encoding one or more activators comprises the nucleotide sequence of SEQ ID NO: 44.

(20) The HSV vector of any one of aspects 17-19, further comprising one or more activation gRNA scaffolds.

(21) The HSV vector of aspect 20, wherein the one or more activation gRNA scaffolds comprise the nucleotide sequence of SEQ ID NO: 37.

(22) A pharmaceutical composition comprising the HSV vector of any one of aspects 1-21 and a pharmaceutically acceptable carrier.

(23) A pharmaceutical composition comprising (a) a first herpes simplex virus (HSV) vector comprising one or more polynucleotides encoding a Cas nuclease and (b) a second HSV vector comprising one or more polynucleotides encoding (i) at least one guide RNA comprising a guide sequence that hybridizes to a target sequence and (ii) one or more repressors.

(24) The pharmaceutical composition of aspect 23, wherein the one or more repressors are selected from the group consisting of Hp1a, Krab, MeCP2, and combinations thereof.

(25) The pharmaceutical composition of aspect 23 or 24, wherein the second HSV vector comprises the nucleotide sequence of SEQ ID NO: 42 or SEQ ID NO: 43.

(26) The pharmaceutical composition of any one of aspects 23-25, wherein the second HSV vector further comprises one or more repression gRNA scaffolds.

(27) The pharmaceutical composition of aspect 26, wherein the one or more repression gRNA scaffolds comprise the nucleotide sequence of SEQ ID NO: 36.

(28) A pharmaceutical composition comprising (a) a first herpes simplex virus (HSV) vector comprising one or more polynucleotides encoding a Cas nuclease and (b) a second HSV vector comprising one or more polynucleotides encoding (i) at least one guide RNA comprising a guide sequence that hybridizes to a target sequence and (ii) activators.

(29) The pharmaceutical composition of aspect 28, wherein the one or more activators are selected from the group consisting of VP64, p65, Rta, HSF1, and combinations thereof.

(30) The pharmaceutical composition of aspect 28 or 29, wherein the second HSV vector comprises the nucleotide sequence of SEQ ID NO: 44.

(31) The pharmaceutical composition of any one of aspects 28-30, wherein the second HSV vector further comprises one or more activation gRNA scaffolds.

(32) The pharmaceutical composition of aspect 31, wherein the one or more activation gRNA scaffolds comprise the nucleotide sequence of SEQ ID NO: 37.

(33) A multiplexed CRISPR-based circuit comprising:

(a) one or more guide RNAs (gRNAs) comprising (i) one or more guide sequences complementary to a portion of target sequence and (ii) one or more gRNA scaffolds;

(b) a nucleotide sequence encoding one or more repressors or one or more activators; and

(c) a nucleotide sequence encoding a Cas nuclease.

(34) The multiplex CRISPR-based circuit of aspect 33, wherein nucleotide sequence encoding the Cas nuclease is packaged in a vector for DNA-based viral delivery.

(35) The multiplex CRISPR-based circuit of aspect 33 or 34, wherein the Cas nuclease is Cas9 nuclease.

(36) The multiplex CRISPR-based circuit of any one of aspects 33-35, wherein the nucleotide sequence encoding Cas9 protein comprises the nucleotide sequence of SEQ ID NO: 38, SEQ ID NO: 39, SEQ ID NO: 40, or SEQ ID NO: 41.

(37) The multiplex CRISPR-based circuit of any one of aspects 33-36, wherein the vector is a herpes simplex virus (HSV) vector.

(38) The multiplex CRISPR-based circuit of any one of aspects 33-37, wherein the target sequence is selected from the group consisting of SCN9A, MyD88, PENK, GAD1, KCNA2, and combinations thereof.

(39) The multiplex CRISPR-based circuit of any one of aspects 33-38, wherein the one or more guide sequences is selected from the group consisting of:

(a) one or more of SEQ ID NOs: 1-7;

(b) one or more of SEQ ID NOs: 8-12;

(c) one or more of SEQ ID NOs: 13-20;

(d) one or more of SEQ ID NOs: 21-28;

(e) one or more of SEQ ID NOs: 29-35; and

(f) combinations thereof.

(40) The multiplex CRISPR-based circuit of any one of aspects 33-39, wherein HSV vector comprises at least two guide RNAs.

(41) The multiplex CRISPR-based circuit of any one of aspects 33-40, wherein HSV vector comprises at least three guide RNAs.

(42) The multiplex CRISPR-based circuit of any one of aspects 33-41, wherein HSV vector comprises at least four guide RNAs.

(43) The multiplex CRISPR-based circuit of any one of aspects 33-42, wherein HSV vector comprises at least five guide RNAs.

(44) The multiplex CRISPR-based circuit of any one of aspects 33-43, wherein the one or more guide RNAs hybridize to different target sequences.

(45) The multiplex CRISPR-based circuit of any one of aspects 33-44, wherein the one or more repressors are selected from the group consisting of Hp1a, Krab, MeCP2, and combinations thereof.

(46) The multiplex CRISPR-based circuit of any one of aspects 33-45, wherein the one or more activators are selected from the group consisting of VP64, p65, Rta, HSF1, and combinations thereof.

(47) The multiplex CRISPR-based circuit of any one of aspects 33-46, wherein the one or more gRNA scaffolds comprise an aptamer target site specific for an RNA binding protein.

(48) The multiplex CRISPR-based circuit of aspect 47, wherein the one or more repressors or one or more activators are fused to the RNA binding protein.

(49) The multiplex CRISPR-based circuit of aspect 48, wherein the one or more repressors or one or more activators fused to the RNA binding protein are encoded by the nucleotide sequence of SEQ ID NO: 42, SEQ ID NO: 43, or SEQ ID NO: 44.

(50) A pharmaceutical composition comprising the multiplex CRISPR-based circuit of any one of aspects 33-49 and a pharmaceutically acceptable carrier.

(51) A method for ameliorating pain in a subject, the method comprising administering to the subject a therapeutically effective amount of the HSV vector of any one of aspects 1-21, the pharmaceutical composition of any one of aspects 22-32 and 50, or the multiplex CRISPR-based circuit of any one of aspects 33-49, thereby ameliorating pain in the subject.

(52) The method of aspect 51, wherein the HSV vector, the pharmaceutical composition, or the multiplex CRISPR-based circuit is injected subcutaneously.

(53) The method of aspect 51 or 52, wherein the HSV vector, the pharmaceutical composition, or the multiplex CRISPR-based circuit targets nerve cells.

(54) The method of any one of aspects 51-53, wherein the HSV vector, the pharmaceutical composition, or the multiplex CRISPR-based circuit is administered to the subject more than once.

The following example further illustrates aspects of the invention but, of course, should not be construed as in any way limiting its scope.

### EXAMPLE 1

This example demonstrates aptamer-mediated CRISPR epigenetic modulation in vitro.

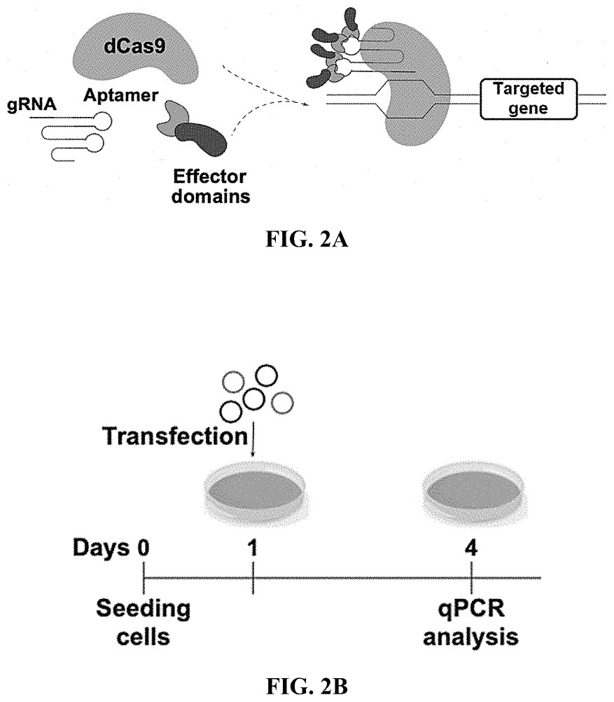

Mouse neuroblastoma (N2A) cells were transfected with either 14 nucleotide or 20 nucleotide gRNAs that hybridize to a portion of target gene involved in nociceptive processing (Scna9, Myd88, Penk, GAD1, or Kcna2) together with dCas9 plasmid and effector cassette (e.g., MS2-HP1a-KRAB or PCP-VP64-P65AD-BRLF-1AD). Expression levels of the targeted gene's mRNA were analyzed using qRT-PCR three days post transfection (see FIGS. 2A and 2B).

dCas9 refers to a nuclease-dead Cas9 (dCas9), which remains competent for DNA binding but lacks endonuclease activity, which is generated by mutating the amino acids critical for DNA catalysis as described in Yeo et al., Nat. Methods, 15(8): 611-616 (2018)). dCas9 corresponds to the nucleotide sequence of SEQ ID NO: 38.

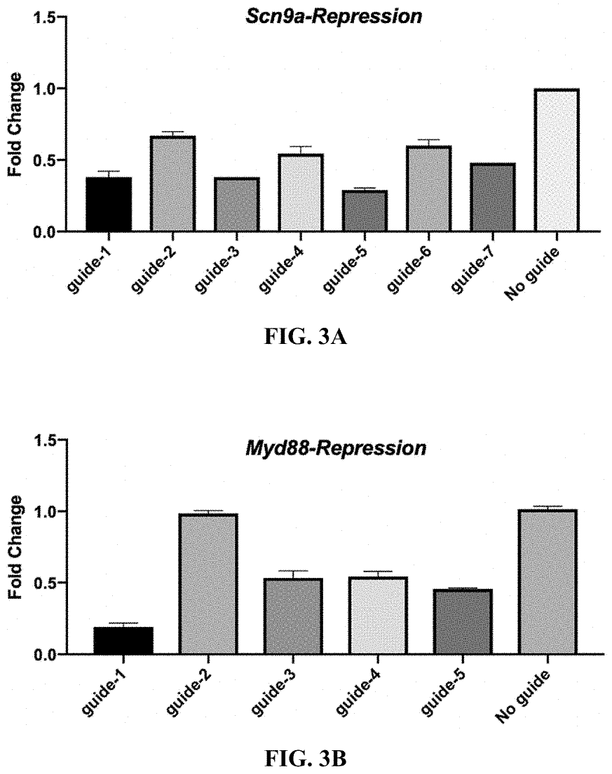

To evaluate endogenous targeted gene expression using CRISPR-mediated repression, N2A cells were transfected with the gRNAs (corresponding to SEQ ID NOs: 1-12) along with dCas9 and MS2-HP1a-KRAB (corresponding to SEQ ID NO: 42) on two separate cassettes. FIG. 3A shows the fold changes of mRNA of Scna9 relative to the No Guide group (no gRNA). FIG. 3B shows the fold changes of mRNA of Myd88 relative to the No Guide group (no gRNA).

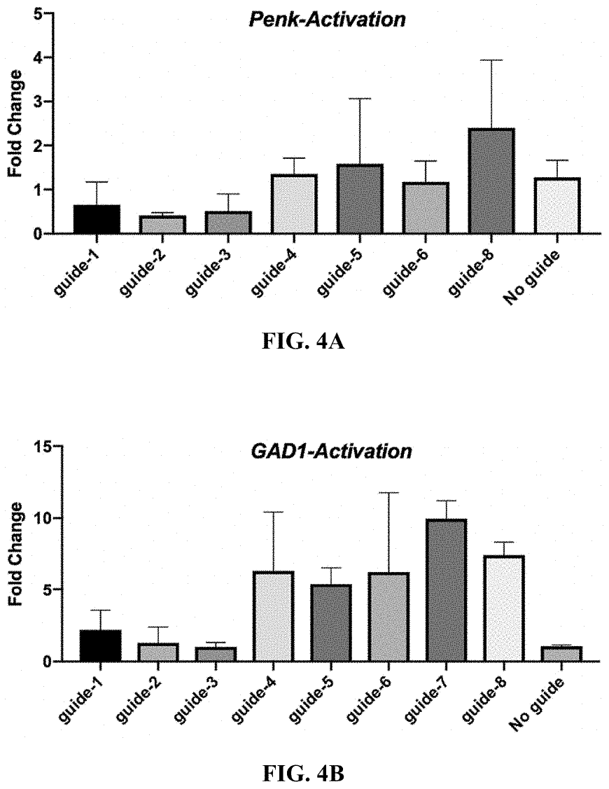

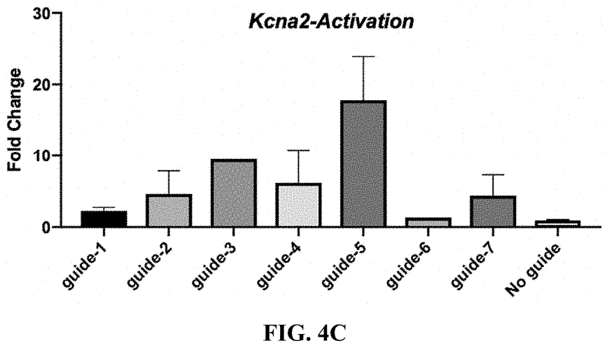

To evaluate endogenous targeted gene expression using CRISPR-mediated activation, N2A cells were transfected with the gRNAs (corresponding to SEQ ID NOs: 13-35) along with dCas9 and PCP-VPR (PCP-VP64-P65AD-BRLF-1AD) on two separate cassettes. Fold changes of mRNA of Penk (FIG. 4A), GAD1 (FIG. 4B), and Kcna2 (FIG. 4C) were quantified relative to the No Guide group (no gRNA).

These results demonstrate that aptamer-mediated CRISPR epigenetic modulation can be used to modulate the expression of genes involved in nociceptive processing for the amelioration of pain and the management of pain in subjects.

### EXAMPLE 2

This example demonstrates a strategy for the management of pain and associated symptoms in a mouse model using CRISPR epigenetic modulation.

Na_{V}1.7 (SCN9A) gain of function mutations yield anomalous hyperpathic states. Expression of Kv1.2 is down-regulated during peripheral nerve injury, partly through activation of Kv1.2 antisense RNA (AS). Preventing this down-regulation can treat neuropathic pain (Guedon et al., Molecular Pain, 11(27): doi:10.1186/s12990-015-0018-1 (2015)).

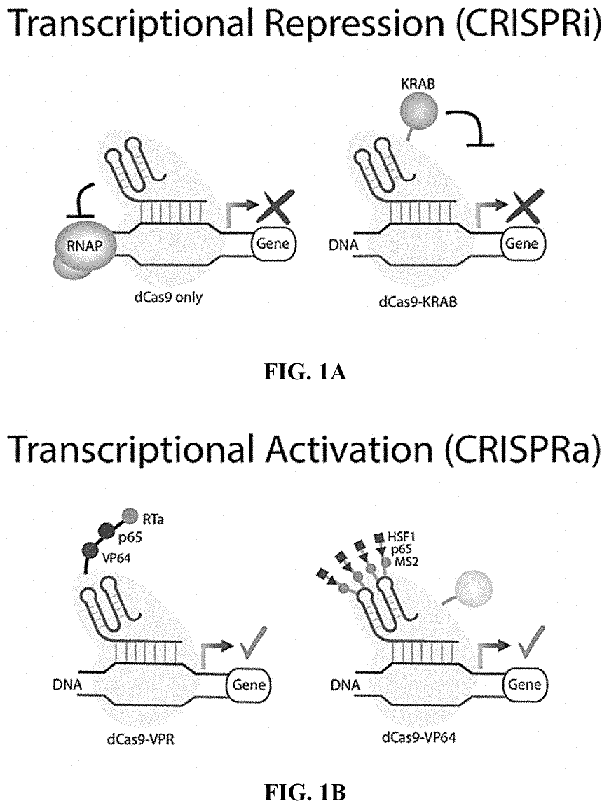

Therefore, repression of the expression of Na_{V}1.7 can be targeted through recruitment of the CRISPR repressor complex to the endogenous promoter region (Dib-Hajj et al., Nat. Rev. Neurosci., 14: 49-62 (2013)) (see FIG. 1A). Additionally, expression of Kv1.2 anti-sense RNA can be repressed by use of a CRISPR repressor to activate Kᵥ1.2 (Zhao et al., Nat. Neurosci., 16(8): 1024-31 (2013)) (see FIG. 1A).

Alternatively, the expression of Kᵥ1.2 and proenkephalin can be activated through recruitment of a CRISPR activator complex to the endogenous promoter (see FIG. 1B).

MyD88 signaling in dorsal root ganglion (DRG) contributes to persistent neuroinflammation (Liu et al., Sci Rep., 6: 28188 (2016)). Therefore, to reduce inflammation, the expression of MyD88 (NFkB pathway) in DRG is reduced through recruitment of CRISPR repressor complex to the endogenous promoter (Moghadam et al., Nature Cell Biology, 22: 1143-1154 (2020), and Liu et al., Sci Rep., 6: 28188 (2016)) (see FIG. 1A). MyD88 repression by enhanced CRISPR repressor has been shown to modulate inflammation using adeno-associated viral vectors (Moghadam et al., Nature Cell Biology, 22: 1143-1154 (2020).

To induce pain in a mouse model, carrageenan is subcutaneously injected into mice hind paws. Alternatively, a hotplate is used on mice hind paws to induce pain.

The HSV vector, multiplexed CRISPR-based circuit, or pharmaceutical composition thereof of aspects of the invention is subcutaneously injected in the mice hind paws (Guedon et al., Molecular Pain, 11(27): doi: 10.1186/s12990-015-0018-1 (2015)). As described herein, the HSV vector, multiplexed CRISPR-based circuit, or pharmaceutical composition thereof comprises at least one guide RNA comprising a guide sequence that hybridizes to a target sequence corresponding to Na_{V}1.7 (SCN9A), MyD88, PENK, KCNA2, and combinations thereof

At timed intervals, edema and hyperalgesia are monitored, after which the limb and spinal cord are harvested and downstream assays (e.g., qPCR) are performed to determine the expression of the target sequences.

This example demonstrates that pain and associated symptoms can be ameliorated in a mouse model using CRISPR epigenetic modulation.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A recombinant herpes simplex virus (HSV) vector comprising one or more polynucleotides encoding (a) at least one guide RNA comprising a guide sequence that hybridizes to a target sequence, and (b) a Cas nuclease.

2. The HSV vector of claim 1, wherein the HSV vector is HSV-1.

3. The HSV vector of claim 1, wherein the Cas nuclease is Cas9 nuclease.

4. The HSV vector of claim 3, wherein the one or more polynucleotides encoding Cas9 protein comprise the nucleotide sequence of SEQ ID NO: 38, SEQ ID NO: 39, SEQ ID NO: 40, or SEQ ID NO: 41.

5. The HSV vector of claim 1, wherein the target sequence is selected from the group consisting of SCN9A, MyD88, PENK, GAD1, KCNA2, and combinations thereof.

6. The HSV vector of claim 1, wherein the guide sequence is selected from the group consisting of:
(a) one or more of SEQ ID NOs: 1-7;
(b) one or more of SEQ ID NOs: 8-12;
(c) one or more of SEQ ID NOs: 13-20;
(d) one or more of SEQ ID NOs: 21-28;
(e) one or more of SEQ ID NOs: 29-35; and
(f) combinations thereof.

7. The HSV vector of claim 1, wherein HSV vector comprises at least two guide RNAs.

8. The HSV vector of claim 1, further comprising one or more polynucleotides encoding one or more repressors.

9. The HSV vector of claim 8, wherein the one or more repressors are selected from the group consisting of Hp1a, Krab, MeCP2, and combinations thereof.

10. The HSV vector of claim 8, wherein the one or more polynucleotides encoding one or more repressors comprise the nucleotide sequence of SEQ ID NO: 42 or SEQ ID NO: 43.

11. The HSV vector of claim 8, further comprising one or more repression gRNA scaffolds.

12. The HSV vector of claim 1, further comprising one or more polynucleotides encoding one or more activators.

13. The HSV vector of claim 12, wherein the one or more activators are selected from the group consisting of VP64, p65, Rta, HSF1, and combinations thereof

14. The HSV vector of claim 12, wherein the one or more polynucleotides encoding one or more activators comprises the nucleotide sequence of SEQ ID NO: 44.

15. The HSV vector of claim 12, further comprising one or more activation gRNA scaffolds.

16. The HSV vector of claim 15, wherein the one or more activation gRNA scaffolds comprise the nucleotide sequence of SEQ ID NO: 37.

17. A pharmaceutical composition comprising the HSV vector of claim 1 and a pharmaceutically acceptable carrier.

18. A multiplexed CRISPR-based circuit comprising:
(a) one or more guide RNAs (gRNAs) comprising (i) one or more guide sequences complementary to a portion of target sequence and (ii) one or more gRNA scaffolds;
(b) a nucleotide sequence encoding one or more repressors or one or more activators; and
(c) a nucleotide sequence encoding a Cas nuclease.

19. A pharmaceutical composition comprising the multiplex CRISPR-based circuit of claim 18 and a pharmaceutically acceptable carrier.

20. A method for ameliorating pain in a subject, the method comprising administering to the subject a therapeutically effective amount of the HSV vector of claim 1, thereby ameliorating pain in the subject.


SEQUENCE LISTING (44 sequences)

SEQ ID NO: 1 - DNA, 20 bp, Artificial Sequence
  Synthetic
gacagtgggcaggattgaaa

SEQ ID NO: 2 - DNA, 19 bp, Artificial Sequence
  Synthetic
gcaggtgcactcaccgggt

SEQ ID NO: 3 - DNA, 20 bp, Artificial Sequence
  Synthetic
gagctcagggagcatcgagg

SEQ ID NO: 4 - DNA, 20 bp, Artificial Sequence
  Synthetic
gctaggagtccgggataccc

SEQ ID NO: 5 - DNA, 20 bp, Artificial Sequence
  Synthetic
gcgacgcggttggcagccga

SEQ ID NO: 6 - DNA, 20 bp, Artificial Sequence
  Synthetic
gacccatgtaacaactccac

SEQ ID NO: 7 - DNA, 20 bp, Artificial Sequence
  Synthetic
gtgtatattgttgaacccgt

SEQ ID NO: 8 - DNA, 14 bp, Artificial Sequence
  Synthetic
gcgagcgtactgga

SEQ ID NO: 9 - DNA, 14 bp, Artificial Sequence
  Synthetic
tgcgggtcctgcac

SEQ ID NO: 10 - DNA, 14 bp, Artificial Sequence
  Synthetic
gccgcagacatggc

SEQ ID NO: 11 - DNA, 14 bp, Artificial Sequence
  Synthetic
gggatcccacgcgg

SEQ ID NO: 12 - DNA, 14 bp, Artificial Sequence
  Synthetic
gggcaggcaaccct

SEQ ID NO: 13 - DNA, 20 bp, Artificial Sequence
  Synthetic
ggctcgggagcaggcgcttg

SEQ ID NO: 14 - DNA, 20 bp, Artificial Sequence
  Synthetic
gcgccaaggcacgggcgact

SEQ ID NO: 15 - DNA, 20 bp, Artificial Sequence
  Synthetic
gcgttcgtccaactttctcg

SEQ ID NO: 16 - DNA, 20 bp, Artificial Sequence
  Synthetic
ggttgcttagggcaagattc

SEQ ID NO: 17 - DNA, 20 bp, Artificial Sequence
  Synthetic
ggagggatcaatttcgggct

SEQ ID NO: 18 - DNA, 20 bp, Artificial Sequence
  Synthetic
gctgtagataggtgccactc

SEQ ID NO: 19 - DNA, 20 bp, Artificial Sequence
  Synthetic
gcacgcggccccgagggtgg

SEQ ID NO: 20 - DNA, 20 bp, Artificial Sequence
  Synthetic
gactctggggaggacacgaa

SEQ ID NO: 21 - DNA, 20 bp, Artificial Sequence
  Synthetic
gggggaggcgggggcggagt

SEQ ID NO: 22 - DNA, 20 bp, Artificial Sequence
  Synthetic
ggagaggcggccgctgggct

SEQ ID NO: 23 - DNA, 20 bp, Artificial Sequence
  Synthetic
ggaggcggggacccgctcgg

SEQ ID NO: 24 - DNA, 20 bp, Artificial Sequence
  Synthetic
gatcacgaggccgttgctcg

SEQ ID NO: 25 - DNA, 19 bp, Artificial Sequence
  Synthetic
gattataaatccagctcac

SEQ ID NO: 26 - DNA, 20 bp, Artificial Sequence
  Synthetic
gcgcgggggcagctcgtccg

SEQ ID NO: 27 - DNA, 20 bp, Artificial Sequence
  Synthetic
gggtttgcgaccagaaagga

SEQ ID NO: 28 - DNA, 20 bp, Artificial Sequence
  Synthetic
gggcagtcagacacctgcaa

SEQ ID NO: 29 - DNA, 20 bp, Artificial Sequence
  Synthetic
gctcgccgccgccgccaagc

SEQ ID NO: 30 - DNA, 20 bp, Artificial Sequence
  Synthetic
gttcgctgcggggccaggtg

SEQ ID NO: 31 - DNA, 20 bp, Artificial Sequence
  Synthetic
gtaggttttgtcaaagaatg

SEQ ID NO: 32 - DNA, 20 bp, Artificial Sequence
  Synthetic
gttctttgcggtgggggtcc

SEQ ID NO: 33 - DNA, 20 bp, Artificial Sequence
  Synthetic
gctctgcgcggagttttgtt

SEQ ID NO: 34 - DNA, 19 bp, Artificial Sequence
  Synthetic
gaactcgaattaaaggggc

SEQ ID NO: 35 - DNA, 19 bp, Artificial Sequence
  Synthetic
gggttagatccctgtgtcc

SEQ ID NO: 36 - DNA, 136 bp, Artificial Sequence
  Synthetic
  misc_feature (19)..(35): Ms2 stem loop
  misc_feature (89)..(105): Ms2 stem loop
gttttagagctaggccaacatgaggatcacccatgtctgcagggcctagcaagttaaaataaggctagtccgttatcaacttggccaacatgaggatcacccatgtctgcagggccaagtggcaccgagtcggtgc

SEQ ID NO: 37 - DNA, 156 bp, Artificial Sequence
  Synthetic
  misc_feature (19)..(43): PCP stem loop
  misc_feature (101)..(125): PCP stem loop
gttttagagctaggccaataaggagtttatatggaaacccttatctgcagggcctagcaagttaaaataaggctagtccgttatcaacttggccaaggagtaaggagtttatatggaaacccttatctgcagggccaagtggcaccgagtcggtgg

SEQ ID NO: 38 - DNA, 4140 bp, Artificial Sequence
  Synthetic
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagacaatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctgggctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtcccccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtga

SEQ ID NO: 39 - DNA, 4273 bp, Artificial Sequence
  Synthetic
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacggtattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattcttgaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaag

SEQ ID NO: 40 - DNA, 4272 bp, Artificial Sequence
  Synthetic
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaaccttattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattggtgaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaa

SEQ ID NO: 41 - DNA, 4270 bp, Artificial Sequence
  Synthetic
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacggtattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattggtgaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagt

SEQ ID NO: 42 - DNA, 1134 bp, Artificial Sequence
  Synthetic
  misc_feature (33)..(43): Ms2
  misc_feature (486)..(875): Hp1a
  misc_feature (907)..(1134): krab
ccaccatgggacctaagaaaaagaggaaggtggcggccgctgactacaaggatgacgacgataaatctagaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgaggccagccccaagaagaagaggaaggtgagtggtggaggaagtggcgggtcagggtcgtccagccccaagaaaaaacggaaagtggaggcatcaatgaaggagggagaaaacaacaaacccagggagaaaagtgaagggaataagagaaaaagctccttctctaacagtgcagacgatatcaagtccaagaaaaagcgggagcagtctaatgacattgctaggggcttcgagagaggactggagccagaaaaaatcattggggcaaccgacagctgcggcgatctgatgtttctcatgaaatggaaggacacagatgaggccgacctggtgctcgccaaagaagctaacgtgaagtgtccccagatcgtcattgctttttacgaggaaaggctcacctggcacgcatatcctgaggatgccgaaaacaaggagaaggaatcagctaagagcctcgggaggtggttcgggtggctctggatcaatggacgcgaaatcacttacggcatggtcgagaacactggttacgttcaaggacgtgtttgtggactttacacgtgaggagtggaaattgctggatactgcgcaacaaattgtgtatcgaaatgtcatgcttgagaattacaagaacctcgtcagtctcggataccagttgacgaaaccggatgtgatccttaggctcgaaaagggggaagaaccttggctggtatga

SEQ ID NO: 43 - DNA, 1634 bp, Artificial Sequence
  Synthetic
  misc_feature (33)..(431): MS2
  misc_feature (486)..(710): krab
  misc_feature (741)..(1634): Mecp2
ccaccatgggacctaagaaaaagaggaaggtggcggccgctgactacaaggatgacgacgataaatctagaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgaggccagccccaagaagaagaggaaggtgagtggtggaggaagtggcgggtcagggtcgtccatggacgcgaaatcacttacggcatggtcgagaacactggttacgttcaaggacgtgtttgtggactttacacgtgaggagtggaaattgctggatactgcgcaacaaattgtgtatcgaaatgtcatgcttgagaattacaagaacctcgtcagtctcggataccagttgacgaaaccggatgtgatccttaggctcgaaaagggggaagaaccttggctggtatcgggaggtggttcgggtggctctggatcaagcccaaagaagaaacggaaggtggaagcctcagtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcatga

SEQ ID NO: 44 - DNA, 1956 bp, Artificial Sequence
  Synthetic
  misc_feature (1)..(363): PCP
  misc_feature (364)..(549): VP64
  misc_feature (586)..(1368): p65 AD
  misc_feature (1387)..(1956): BRLF1 AD
tccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt